(19) United States
(12) Patent Application Publication
(10) Pub. No.: US 2010/0063453 A1
(43) Pub. Date: Mar. 11, 2010

(54) Gateway Device

(76) Inventors: Julie Grundtvig Theander, Olstykke (DK); Brian Boyum Hagedorn, Soro (DK)

Correspondence Address: BRINKS HOFER GILSON & LIONE, P.O. BOX 10395, CHICAGO, IL, 60610, US

(21) Appl. No.: 12/524,928

(22) PCT Filed: Feb. 1, 2008
(86) PCT No.: PCT/EP08/51276

§ 371 Date: Nov. 10, 2009

Related U.S. Application Data
(60) Provisional application No. 60/899,143, filed on Feb. 2, 2007.
(60) Provisional application No. 60/899,075, filed on Feb. 2, 2007.
(60) Provisional application No. 60/899,062, filed on Feb. 2, 2007.

(30) Foreign Application Priority Data
Feb. 2, 2007 (DK) PA 2007 00179
Feb. 2, 2007 (DK) PA 2007 00185
Feb. 2, 2007 (DK) PA 2007 00191

(51) Int. Cl. A61M 25/02; A61M 25/16; A61M 5/00

(52) U.S. Cl. 604/180; 604/533; 604/244

(57) ABSTRACT

A gateway device for the delivery of a drug, said device comprising a cannula (40) for delivery of said drug, a housing (10) having a base (20) adapted for being placed against the skin of a patient, and a receiving area (30) for receiving a coupling portion (2) of a drug supply device (4) for supplying said drug to said gateway device, said receiving area (30) including a hollow tubular coupling (50) having a free end (52) and being for engaging said coupling portion, said hollow tubular coupling (50) being arranged for delivery of said drug to said patient via said cannula (40). The cannula (40) is secured to said housing (10) to allow said cannula (40) to project from said housing (10), wherein a hollow tubular member with a first end portion and a second end portion defines said cannula and said tubular coupling.

The present invention relates to a gateway device for the delivery of a drug, the device comprising a cannula for delivery of said drug, a housing having a base adapted for being placed against the skin of a patient, and a receiving area for receiving a coupling portion of a drug supply device for supplying the drug to the gateway device, the receiving area including a hollow tubular coupling having a free end and being for engaging the coupling portion, the hollow tubular coupling being arranged for delivery of said drug to said patient via the cannula, the cannula being secured to the housing to allow the cannula to project from said housing.

Conventionally, gateway devices for subcutaneous delivery of a drug require an insertion needle which is passed through the cannula and which is used for placing the gateway device on the patient. This involves the problem that a complicated gateway structure with a separate membrane or septum is required for sealing off the entry port of the insertion needle.

The above problem is solved through the invention by providing a hollow tubular member with a first end portion and a second end portion that defines the cannula and the tubular coupling such the hollow tubular member not only serves as a cannula for delivery of drug but also serves as an insertion needle allowing the user to use the first end portion for inserting the gateway device into the skin whereby no separate insertion needle is required for placing the gateway device. Hence, the first end portion serves the dual function of a cannula and of an insertion needle for the first time placement of the device, the free end of the tubular coupling preferably being adapted for piercing or penetrating a septum of the coupling portion.

Also claimed is a novel insertion device for placing a gateway assembly of the general type including a housing having a base adapted for being placed against the skin of a patient and a cannula for delivery of a drug to said patient, the cannula being secured to said housing to allow the cannula to project from said housing, preferably from said base, an insertion needle of the insertion device being receivable by said cannula.

Embodiments of the invention will now be discussed with reference to the drawings.

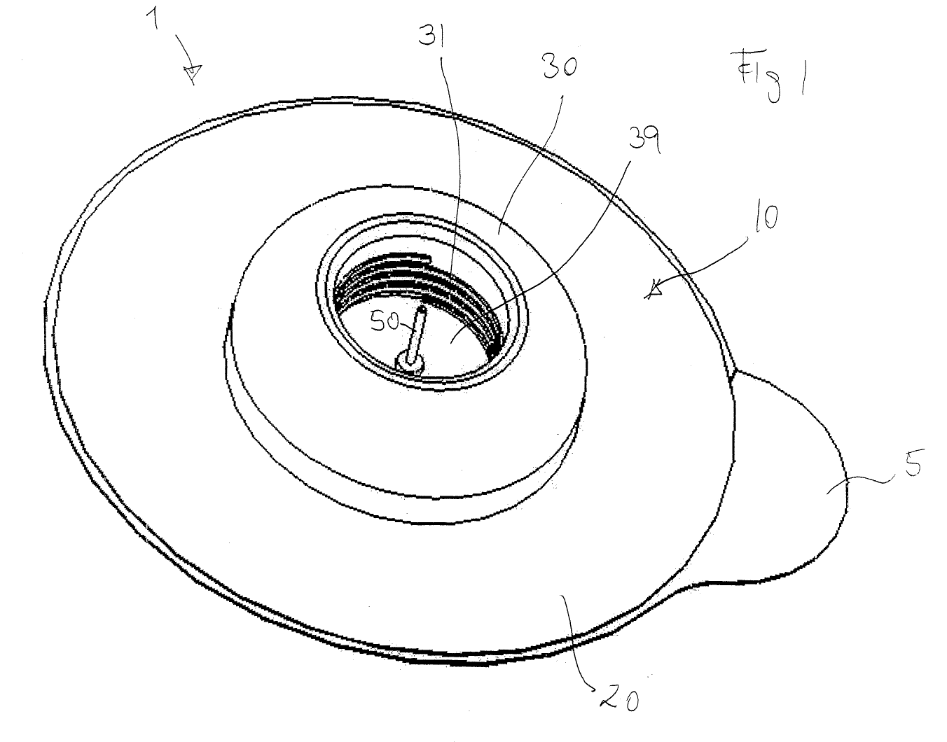

FIG. 1 is a perspective view seen from above of one embodiment of the device according to the invention,

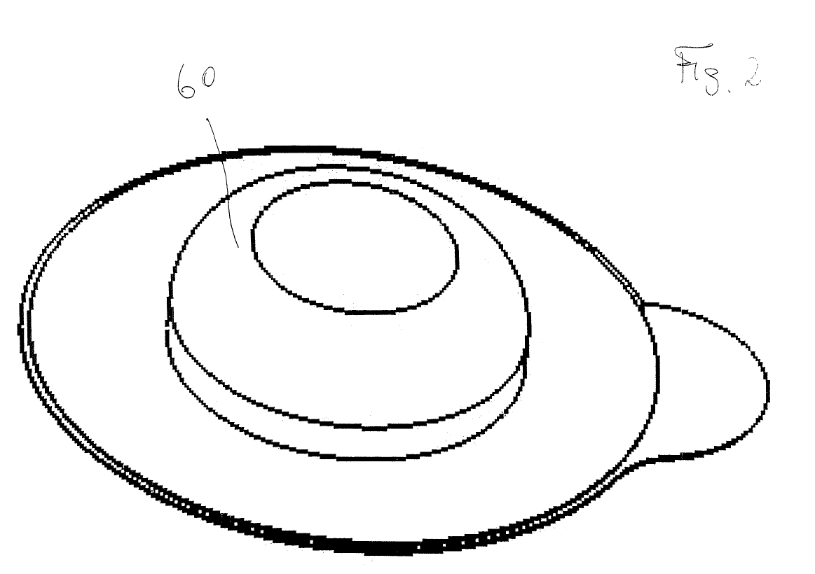

FIG. 2 is a view similar to FIG. 1, with a cap mounted on the device,

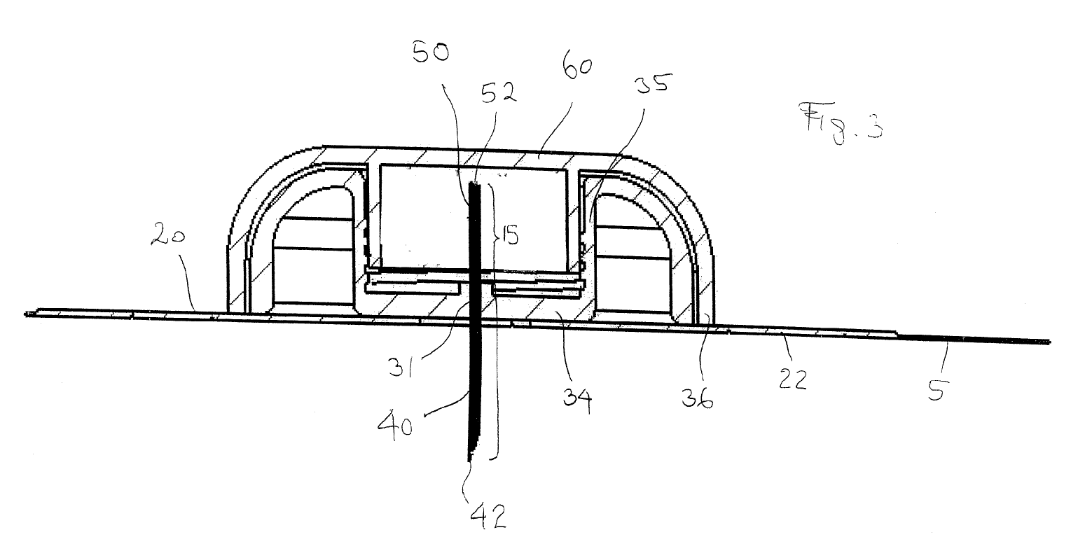

FIG. 3 is a cross-sectional view of the device as shown in FIG. 2,

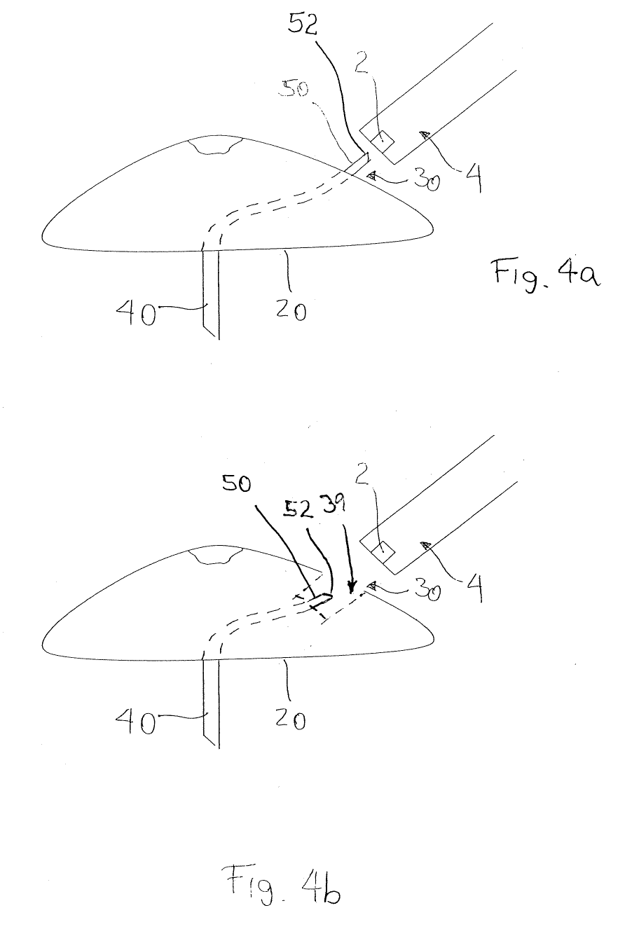

FIGS. 4a and 4b are schematic side views of second and third embodiments of the device, and

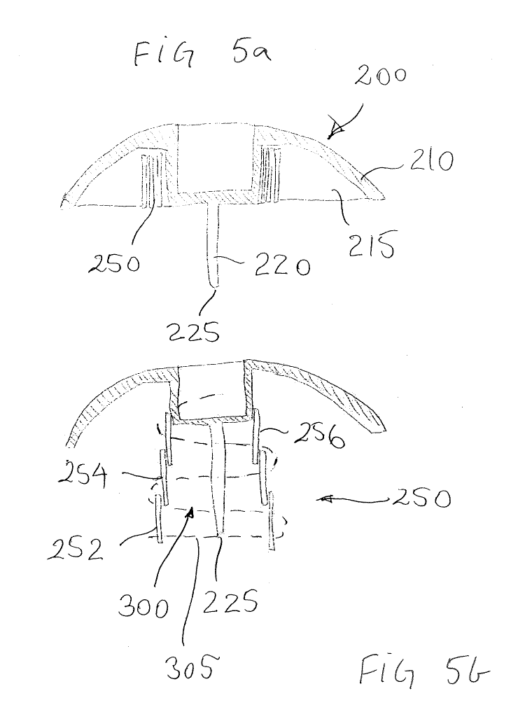

FIG. 5a and FIG. 5b are side views of an inserting device shown in a needle non-protecting and a needle protecting state, respectively.

In use of a gateway device of the invention, after placement of the gateway device on a patient, the gateway device is coupled to a drug supply device that has a coupling portion suitable for coupling the drug supply device to the gateway to supply a drug to the patient via the gateway device.

FIG. 1 shows a first embodiment of a gateway device according to the invention. The gateway device 1 comprises a housing 10 with a base 20 with a lower surface which in use of the gateway device 1 is placed and maintained in position against the skin of a patient to which the drug, such as insulin, is to be administered. The housing 10 may be molded in its entirety from a plastics material, or the device preferably may have an upper housing part molded of a plastics material and to which a flexible sheet lower housing part defining the base 20 may be attached, such as by way of an adhesive. The base 20 lower surface preferably has an adhesive layer covered by a releasable protective sheet 5.

The gateway device 1 has a cannula 40 for delivery of the drug, the cannula 40 with free end 42 being secured to the housing to allow the cannula 40 to project from the housing 10, preferably such that the cannula 40 extends from the lower surface 2 (see FIG. 3) of the base, perpendicularly or at an angle thereto; alternatively the cannula 40 may extend from other parts of the housing.

The housing 10 also includes a receiving area 30 with a central recess 39 for receiving the coupling portion of the aforementioned drug supply device, and the receiving area 30 centrally therein has a hollow tubular coupling 50 that has a free end 52 and is for engaging the coupling portion of the drug supply device. Often the coupling portion of the drug supply device will have a septum, and when the free end 52 of the hollow tubular coupling penetrates the septum by the user inserting the drug supply device coupling portion into the recess 39 the drug will flow to the patient via the cannula 40 which previously has been placed subcutaneously.

The free end 52 of the tubular coupling 50 is preferably sharpened allowing for an easy penetration of the septum, and the tubular coupling 50 may not project beyond the recess 39. As shown in FIG. 2, the device preferably includes a releasable and repositionable cap or cover 60, which may define a microbial barrier, for covering the sharpened free end 52 of the tubular coupling 50 in order to protect the user against inadvertent sticking himself on the tubular coupling 50. The cover 60 is preferably molded from a plastics material and may be connected to the housing 10 in any appropriate manner, such as by way of a screw-thread 31 as illustrated schematically in FIG. 1, or by cooperating engagement means providing for a snap-engagement between the cover 60 and the housing 10. Alternatively a flexible sheet cover 60 may be adhered to the housing 10.

FIG. 3 shows the device with cover 60, a circumferential wall 35 defining the recess 39 and being integral with a recess bottom wall 34 and an outer circumferential wall 36.

The hollow tubular coupling 50 is defined by a first end portion of a hollow tubular member while the cannula 40 is defined by the opposite, second end portion of the hollow tubular member. The hollow tubular member is referenced by numeral 15 in FIG. 3, and the tubular member 15 has a middle portion fixed in the housing 10 in an area of the recess bottom wall 34 designated reference numeral 31, to keep the tubular member 15 in position with respect to the housing 10. In use, the drug flows through and along the inside of the hollow tubular member 15 between the ends 42, 52 thereof.

The hollow tubular member 15 serves several purposes. The first end portion of the hollow tubular member 15 not only serves as a cannula for delivery of drug but also serves as an insertion needle allowing the user to use the first end portion for inserting the gateway device 1 into the skin, such that no separate insertion needle is required for placing the device 1. Hence, the first end portion serves the dual function of a cannula and of an insertion needle for the first time placement of the device. The cannula defined by the first end portion may be more flexible than the tubular coupling defined by the second end portion of the hollow tubular member 15, to increase the comfort experienced by a patient using the gateway device 1, and the tip portion or free end 42 of the cannula 40 may be sharpened. To increase flexibility the first end portion of the hollow tubular member 15 may have reduced cross-sectional dimensions, such as wall-thickness, or the first end portion may be formed from a material different from the second end portion. The hollow tubular member 15 may be formed from the same material, such as steel, and may have uniform cross-sectional dimensions along the length, until the sharpened tip-portions 42, 52 thereof.

As will be seen from FIG. 3 the hollow tubular member is preferably a one-piece member defining a non-leaking flow path for drug to be administered, and the tubular member 15 may be straight as in FIG. 3, or may assume a bent or curved configuration as illustrated schematically in FIGS. 4a and 4b where the tubular coupling 50 extends at an angle with respect to the cannula 40 at the free end 52 to allow the drug supply device with coupling portion septum 2 to be supported by the coupling portion receiving area 30 at an angle to the base 20. In FIGS. 4a and 4b the hollow tubular member 15 is shaped as an S to provide a curved flow path between the drug supply device 4 and the patient. In FIG. 4b, a recess 39 is provided to provide lateral support and guiding for the supply device such that the hollow tubular coupling 50 is at less risk of being damaged.

While in FIGS. 1-3 a well-defined receiving area formed as a recess is shown, the invention is by no way limited to the gateway device housing having any specific receiving area defining structure. Thus, in principle the receiving area may simply be a surface area of the housing with the tubular coupling extending upwards therefrom, as shown in FIG. 4a, and the receiving area may or may not be adapted to provide for support for the supply device.

FIG. 5 shows an insertion device 200 for placing a different type gateway assembly (not shown herein), the insertion device including a head structure 210, an insertion needle 220 with a tip portion 225 extending from the head structure 210, and a shield structure 250 for avoiding inadvertent needle sticks, the shield structure 250 including a plurality of segments 252, 254, 256 that are arranged in a telescopic manner such that the shield structure 250 may assume an extended state wherein a first segment 252 is closer to the tip portion 225 and a retracted state wherein said first segment 252 is closer to said head structure 210. The insertion device of FIG. 5 may be used for placing subcutaneous drug delivery assemblies of the type having a housing with a base adapted for being placed against the skin of a patient and a cannula for delivery of a drug to the patient, the cannula being secured to the housing to allow the cannula to project from the housing, preferably from the base, and where the insertion needle is received by the cannula to effect insertion of the cannula, after which the insertion device is withdrawn, thus potentially leaving the insertion needle sharpened tip exposed.

In the device of FIG. 5 at least the first segment 252 is being spring loaded towards said extended state, such as by way of a spring (not shown) or through the structure of the segments, whereby at least the first segment 252 defines an annular wall extending around the insertion needle 220, and the head structure 210 may include a recess 215 for receiving at least one of the segments in the retracted state.

Alternatively, a spring structure, such as a coil spring, may extend around the needle 220 as shown in phantom line in FIG. 5, the spring assuming an extended state wherein a first part 305 of the spring is closer to the tip portion 225 and a retracted state wherein the first part 305 is closer to the head structure and where the spring is compressed. The spring structure may be of plastics material integrally molded with a plastics material head structure 210, and the spring structure may be similar to the spring structure shown in FIG. 21a of US patent application no. 2004204687.

1. A gateway device for the delivery of a drug, said device comprising:
a housing having
a base adapted for being placed against the skin of a patient;
a hollow tubular member having a first end portion defining a cannula and a second end portion defining a tubular coupling, said cannula secured to said housing to allow said cannula to project from said housing for delivery of said drug; and
a receiving area for receiving a coupling portion of a drug supply device for supplying said drug to said gateway device,
said receiving area including the hollow tubular coupling having a free end configured for engaging said coupling portion,
said hollow tubular coupling being arranged for delivery of said drug to said patient via said cannula.

2. The gateway according to claim 1, wherein said free end of said tubular coupling is adapted for penetrating a septum of said coupling portion.

3. The gateway according to claim 1, wherein said cannula is more flexible than said tubular coupling.

4. The gateway according to claim 1, wherein said cannula comprises a plastics material and said tubular coupling comprises a metal.

5. The gateway according to claim 1, wherein said cannula and said tubular coupling comprise the same material.

6. The gateway according to claim 1, wherein said cannula and said hollow tubular coupling together define a non-leaking flow path for said drug.

7. The gateway according to claim 1, wherein said hollow tubular member is a needle.

8. The gateway according to claim 1, wherein said hollow tubular member is substantially straight.

9. The gateway according to claim 1, wherein said hollow tubular member comprises a bent configuration.

10. The gateway according to claim 1, wherein said tubular coupling extends perpendicularly to said base.

11. The gateway according to claim 1, wherein said tubular coupling extends at an angle to said base.

12. The gateway according to claim 1, wherein said housing includes a releasable cover for covering said tubular coupling.

13. The gateway according to claim 12, wherein said cover surrounds said receiving area.

14. The gateway according to claim 12, wherein said cover is secured to said receiving area by a screw-thread or by snap engagement.

15. The gateway according to claim 12, wherein said cover is hinged to said receiving area.

16. The gateway according to claim 12, wherein said receiving area includes a wall structure surrounding said tubular coupling.

17. The gateway device according to claim 16, wherein said cover being is secured to said wall structure.

18. The gateway device according to claim 16, wherein an imaginary extension of said wall structure below said base surrounds said cannula.

19. The gateway according to claim 1, wherein said receiving area includes a recess.

20. The gateway according to claim 1, wherein said tubular coupling does not project outwardly beyond said receiving area.

21. The gateway according to claim 1, said receiving area being integrally formed with said base, such as by molding.

22. The gateway according to claim 1, wherein said base includes an adhesive layer for securing said base to said patient, said device including releasable protection on said adhesive layer.

23. (canceled)

24. The gateway according to claim 1, wherein said hollow tubular member comprises a one-piece member.

25. A system comprising the gateway device of claim 1, and a drug supply device with a coupling portion for supplying said drug to said gateway device, said coupling portion including a septum adapted to be penetrated by said hollow tubular coupling.
